(19) United States
(12) Patent Application Publication
(10) Pub. No.: US 2010/0049009 A1
(43) Pub. Date: Feb. 25, 2010

(54) APPARATUS AND ARRANGEMENT FOR CAPTURING OR EVALUATING MEDICAL DATA ON BOARD A MEANS OF TRANSPORT

(75) Inventor: Andrew MUIRHEAD, Norderstedt (DE)

Correspondence Address: MORRISON & FOERSTER LLP, 1650 TYSONS BOULEVARD, SUITE 400, MCLEAN, VA, 22102, US

(73) Assignee: Lufthansa Technik AG, Hamburg (DE)

(21) Appl. No.: 12/544,126

(22) Filed: Aug. 19, 2009

(30) Foreign Application Priority Data
Aug. 19, 2008 (DE) 102008038312.0

(51) Int. Cl. A61B 5/00; H04W 4/00

(52) U.S. Cl. 600/301; 370/328

(57) ABSTRACT

An apparatus for capturing and/or evaluating medical data on board a means of transport, and a system including the apparatus. The apparatus includes at least one interface configured to capture or input medical data and a device configured to transmit medical data, audio data, or video data from the apparatus to or from a ground station situated outside the means of transport via data transmission means. The apparatus has a first and second mode of operation for transmitting data to or from the ground station. The first mode of operation is designed to use a first bandwidth sufficient for video transmission on the data transmission means. The second mode of operation is designed to use a second bandwidth narrower than the first bandwidth or a discontinuous data transmission mode on the data transmission means.

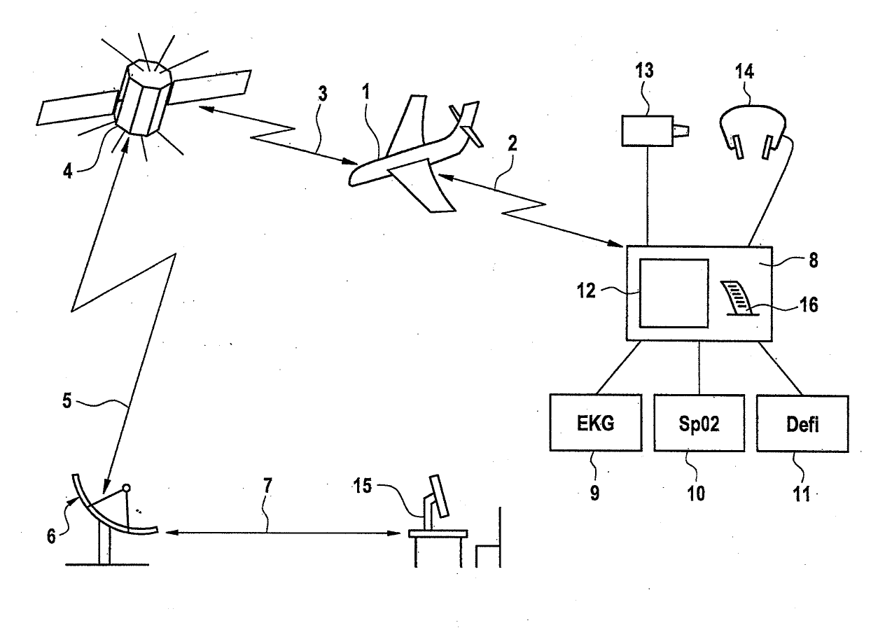

### REFERENCE TO RELATED APPLICATIONS

This application claims the priority of German Patent Application No. 10 2008 038 312.0, filed Aug. 19, 2008, the contents of which are incorporated herein by reference.

### FIELD OF THE INVENTION

The invention relates to an apparatus and an arrangement for capturing or evaluating medical data on board a means of transport.

### BACKGROUND OF THE INVENTION

In remote locations, particularly means of transport such as aircraft, ships or the like, medical emergencies may arise without there being competent medical assistance immediately available. As an example, if a medical emergency arises on board an aircraft in flight, the cabin crew can administer first aid, and it may even be possible to call upon a doctor who is present among passengers on board.

For the cabin crew on an aircraft, it may be difficult to reach a decision as to whether the seriousness of the medical incident or emergency requires an unscheduled landing at the closest airport at which medical assistance is available. Since an unscheduled landing of this kind is associated with high costs and inconvenience to the other passengers, by way of example WO 03/030405 A1 has already disclosed the practice of capturing medical data from the person in question and using what is known as a telemedicine module to transmit said data to a ground station, where they can be evaluated by a doctor. In addition, this telemedicine module can be used to communicate with a doctor on the ground.

### SUMMARY OF THE INVENTION

The invention is based on the object of providing an apparatus and an arrangement of the type cited at the outset which allow efficient and inexpensive communication between the apparatus for capturing or evaluating medical data which is on board and a ground station (what is known as a telemedicine station on the ground).

The apparatus according to the invention has the following features:
a) at least one interface for the capture and/or input of medical data;
b) a device for transmitting medical data and/or audio and/or video data from the apparatus to and/or from a ground station situated outside the means of transport;
c) the apparatus has a first mode of operation for transmitting medical data and/or audio and/or video data from the apparatus to and/or from a ground station situated outside the means of transport, in which it is designed to protect a bandwidth, sufficient for video transmission, on the data transmission means used;
d) the apparatus has a second mode of operation for transmitting medical data and/or audio and/or video data from the apparatus to and/or from a ground station situated outside the means of transport, in which it uses a narrower bandwidth than in the first mode of operation and/or a discontinuous data transmission mode (packet mode) for the data transmission.

First of all, a few terms used within the context of the invention will be explained.

An apparatus for capturing and/or evaluating medical data is a preferably portable apparatus into which it is possible to input medical data via at least one interface and which can transmit these data to a ground station either directly or possibly in a conditioned or evaluated form. The medical data may be particularly measurement data from sensors for vital functions (for example blood pressure, pulse, pulse oximetric data, ECG data or the like), and it is also possible for medical data to be input by an ancillary person using a suitable interface, such as a keyboard, a touchscreen or the like.

The term “on board a means of transport” should be understood to have a broad meaning and therefore covers apparatuses which can be used in earth-borne and/or flying means of transport, preferably including in aircraft. It goes without saying that an apparatus according to the invention can also be used at other locations at which medical assistance is not readily available. Examples which may be mentioned are isolated localities, islands, exploratory platforms in water or the like.

The apparatus according to the invention (subsequently also called telemedicine module) has at least two modes of operation in which it communicates with the ground station in different ways. In a first mode of operation, the data transmission means used for communication with the ground station provide a bandwidth which is sufficient for video transmission from and to the ground station. In this mode of operation, it is possible for cabin crew, for example, to use the telemedicine module to speak directly to a doctor in the ground station (audio transmission), and it is additionally possible to use video transmission to transmit realtime images of the patient, for example, to the ground or it is possible for the doctor in the ground station to transmit instructions for action to the cabin crew and to demonstrate them by video. In this first mode of operation, the apparatus uses a relatively high bandwidth, which can be very expensive when using satellite links in a flying aircraft, for example, and can also adversely affect the use of the relevant satellite link by other users on board the aircraft (for example operating systems in the aircraft which transmit maintenance data, or passengers sending E-mail or working on the Internet), since usually the bandwidth available to all users on board a means of transport in the case of a satellite link is limited.

In line with the invention, the apparatus therefore has a second mode of operation, in which it uses a narrower bandwidth than in the first mode of operation and/or uses a discontinuous data transmission mode (for example packet mode). The invention has recognized that many medical emergencies either do not require or at any rate do not permanently require a broadband connection to the ground which allows transmission of video data in real time. If only audio communication with the ground station is required, for example, only a much smaller bandwidth needs to be used. In addition, a situation may arise in which the cabin crew has administered first aid and the telemedicine module is used merely for further recording of vital data from the patient which need to be transmitted to the ground so as to be available there later for evaluation purposes (for example for the purpose of a preparation and performance of further medical treatment). In such a situation, a narrowband data link or possibly a discontinuous data transmission may likewise suffice. The invention therefore allows broadband data transmission, associated with high costs and an adverse effect on other users, to be used only when the situation calls for it. A suitable bandwidth for the second mode of operation may be 32 kBit/s, for example. When an appropriate compression algorithm is used for audio data, it is sometimes even possible to select even lower bandwidths.

Within the context of the invention, the term ground station denotes a device which can be used to set up a data link to the means of transport and which can be used to connect what is known as a telemedicine service provider to the means of transport. The term telemedicine service provider denotes a device which is suitable for evaluating the transmitted medical data and, when required, can transmit diagnostic or therapeutic instructions or advice on board the means of transport. Usually, such a telemedicine service provider is staffed by one or more doctors.

The interfaces for the capture or input of medical data may comprise connections for sensors for vital functions of the body. Examples of such sensors may include ECG appliances, pulse oximeters, blood pressure gauges, body temperature thermometers or the like. Within the context of the invention, it is also possible for there to be interfaces which the telemedicine module can use to actuate therapeutic devices directly when required. An example which may be mentioned in this context is a defibrillator, which can be actuated or triggered by the telemedicine module either automatically using the ascertained diagnostic data or manually either by operating personnel or by the telemedicine station on the ground.

It is also possible to provide interfaces for the capture and/or input of medical data which capture audio and/or video data or are designed for the input of data by an ancillary person, it being possible for such an input to be effected by a keyboard, a touchscreen or the like, for example. In particular, an interface for a video camera may be provided which can be used to document the state of a patient, for example, or which can be used in the telemedicine station on the ground to check the correct performance of diagnostic or therapeutic measures. In addition, connections for a microphone, a loudspeaker or headphones or possibly a combined headset may be provided which simplify audio communication with the telemedicine station.

Within the context of the invention, it is possible for additional sensors for vital functions of the body to be provided which are not connected to the telemedicine module on line. These may be a customary clinical thermometer, for example, an apparatus for determining blood sugar or the like. These sensors can be used by appropriately trained cabin crew, and the measured values can then be input into the telemedicine module manually.

The data transmission between the telemedicine module and the ground station is usually effected such that first of all a suitable data transmission network in the means of transport is used to transmit to a transmission/reception device in the means of transport. This is followed by a usually wireless transmission (for example by means of a broadband microwave link via satellite) to a ground station. Particularly in the case of rail-borne means of transport, there may also be a wired transmission to the ground station. Within the means of transport, the data transmission from the telemedicine module to the network in the means of transport can either be effected by wire or preferably wirelessly, for example by means of a known wireless local area network (WLAN).

In the first mode of operation, the invention may involve the provision of a bandwidth of at least 64 kBit/s, preferably 64 to 128 kBit/s, more preferably at least 96 kBit/s. Such a bandwidth is usually sufficient to allow video transmission in real time and sufficient quality. If a higher resolution and/or quality for a video transmission in real time is required, an appropriately higher minimum bandwidth may be provided in the first mode of operation.

In the second mode of operation, the invention may involve the provision of a bandwidth of at least 16 kBit/s, preferably at least 32 kBit/s, more preferably 32 to 64 kBit/s.

The invention therefore allows efficient use—adversely affecting other users as little as possible—of the available transmission paths between aircraft and ground. Many satellite transmission links (for example the Inmarsat links known in the prior art) can be booked in what is known as packet mode or circuit mode, for example. In packet mode, the data traffic is paid for on the basis of transmitted volume of data, but there is usually no guarantee of a minimum bandwidth or data transmission rate which is available at all times. In what is known as circuit mode, an agreed bandwidth is provided permanently and exclusively. Usually, the payment is made on the basis of the period of use or the provision of this bandwidth. The circuit mode is therefore usually much more expensive and, for reasons of cost, is used only when a particular minimum transmission quality (for example when transmitting video data) needs to be achieved.

Many satellite transmission links which have been on the market for a relatively long time achieve a maximum data transmission rate between aircraft and ground of 256 to 512 kBit/s in total, for example. If a video data signal (usually required minimum bandwidth 64 kBit/s) is transmitted in packet mode on such a satellite transmission link, delays frequently arise in the data transmission on account of communication conflicts with other users of the satellite transmission link (for example passengers sitting on the same aircraft and communicating using the satellite transmission link), which means that realtime video transmission in sufficient quality is not possible. In this case, video transmission then requires a sidestep to the much more expensive circuit mode. In the narrower bandwidth second mode of operation, on the other hand, the cheaper packet mode would usually suffice. If the second mode of operation is also intended to be effected in circuit mode for safety reasons, it is only necessary to book an appropriately smaller guaranteed bandwidth. This reduces costs and leaves greater portions of the total available bandwidth on the satellite transmission link available for other users.

There are broader bandwidth satellite transmission links which e.g. use the Ku band (12 to 18 GHz) and are provided by the company ViaSat, for example. Such satellite transmission links achieve data transmission rates in the Mbit range, for example 1.8 to 10 Mbit/s. The available bandwidth from the aircraft to the ground is in this case frequently smaller than on the inverse path. With appropriately broadband satellite transmission links, it will frequently be possible within the context of the invention to perform the video data transmission for the first mode of operation in packet mode as well. Under some circumstances, however, it may also make sense in this case to likewise allow operation of the first mode of operation on the satellite transmission link in circuit mode so as to achieve perfect and undelayed video data transmission even when the satellite communication link is at a high utilization level.

The invention may provide for the first mode of operation to turn on automatically when a video transmission is performed. This ensures that the apparatus uses the appropriate high bandwidth for video transmission only when a transmission is actually being effected.

Preferably, the apparatus has an identification device which, when the first mode of operation is being used, identifies it to the operator of the data transmission means used as to the privileged requesting of an appropriate bandwidth. By way of example, what is known as the MAC-ID or the network name of the apparatus according to the invention in the network on the aircraft may be registered with the operator of the satellite network which is used to set up the broadband link between aircraft and ground. When the telemedicine module logs into the WLAN on the aircraft, this is brought to the attention of the operator of the satellite network by means of the transmission/reception device in the aircraft. Said operator or the routing system onboard the aircraft then takes precautions to ensure that the necessary minimum bandwidth can be provided at short notice and in preference for a connection between the relevant aircraft and the ground in the satellite network. If appropriate, this requires other connections existing in this satellite network to have their bandwidth restricted or to be isolated, particularly connections which exist between the relevant aircraft and the ground, such as an Internet connection for passengers on board or connections between operating systems in the aircraft and the ground for the interchange of maintenance data. As soon as the first mode of operation (video transmission) is then turned on, the relevant measures are taken in order to provide the necessary minimum bandwidth immediately and without interruption. If the network was only under low load before the medical emergency occurred, it may also be the case that no measures at all are required in order to ensure the necessary minimum bandwidth. Within the context of the invention, it is also possible for the apparatus to have a manual control element which can be used to prescribe a desired minimum bandwidth manually. In this way, the available bandwidth in the second mode of operation, for example, can be increased manually if the apparatus reacts too slowly for the operator.

In line with the invention, the apparatus may have a third mode of operation in which no data transmission from or to a ground station takes place. In this “standalone” mode of operation, the telemedicine module can be used, for example, for capturing diagnostic data or possibly therapy for relatively simple medical incidents which do not require an emergency connection to a telemedicine service provider.

The apparatus according to the invention may additionally have a device which outputs an identification code for a diagnosed or treated patient. This may be a label printer, for example, which prints a barcode or another label onto a band or the like which can be placed around the patient's wrist. The data transmitted to the ground during the medical incident can be stored in a database there. If the patient is taken to a hospital, for example, after the means of transport has landed or arrived, the code on the label can be read there using a suitable reader, which means that the data stored in the ground station can be rendered accessible to the staff of the respective hospital, for example by the Internet. In this way, the relevant data can be accessed easily, reliably and at the same time securely when further treatment follows the medical incident.

The subject matter of the invention is also an arrangement for capturing or evaluating medical data on board a means of transport which has:
a) an apparatus according to one of claims 1 to 8 on board the means of transport;
b) a network for transmitting the data from the apparatus to a transmission/reception device in the means of transport and vice versa on board the means of transport;
c) a ground station having a transmission/reception device which is designed for data communication with the transmission/reception device in the means of transport;
d) devices in the ground station for data communication with a device for providing telemedical services.

As already outlined within the context of the explanation of the apparatus according to the invention, the network used on board the means of transport can be a wireless network, such as a WLAN; the data communication between means of transport and ground station can preferably be effected using a broadband radio link, such as a microwave link via satellite.

Usually, the ground station will forward the relevant data to a telemedicine service provider. This can be done either using proprietary data lines or using a public network such as the Internet. When public networks are used, the data communication can preferably be effected in encrypted form, since personal data which need to be protected are transmitted in this case.

### BRIEF DESCRIPTION OF THE DRAWINGS

An embodiment of the invention is described below with reference to the drawing, which schematically shows an arrangement according to the invention.

### DETAILED DESCRIPTION OF THE INVENTION

A wireless local area network (WLAN) 2 is installed on board an aircraft 1. The aircraft has a link 3, 5 to a ground station 6 via a satellite 4 while it is in flight. This ground station 6 is connected to the Internet 7.

In normal flight, the satellite link 3, 4, 5 can be used to maintain a continual data link to the ground station 6 and hence to the Internet 7. This link can be used by the passengers in the aircraft 1 for communication, and it can also be used by operating systems in the aircraft for the transmission of maintenance data or the like. The WLAN 2 inside the aircraft is used to connect the terminals of the users to a suitable transmission/reception device (not shown) in the aircraft 1 which allows a link 3 to the communication satellite 4.

If a medical incident occurs on board, the cabin crew can activate a telemedicine module 8, which is usually provided in an emergency case or the like. Following activation, the telemedicine module 8 logs into the WLAN 2 in the aircraft 1 and is identified there by means of its MAC-ID. This identification prompts the transmission/reception device in the aircraft 1 to report the occurrence of a medical incident to the ground station 6 via the satellite 4. The operator of the satellite link 3, 4, 5 then ensures that from this point in time onward either a sufficient bandwidth also for transmitting video data in real time via the link 3, 4, 5 is immediately provided or precautions are taken to ensure that this bandwidth can be provided immediately if the first mode of operation (video transmission) is actually activated on the telemedicine module 8.

The cabin crew can set up sensors for vital functions of the body on the patient and connect them to the telemedicine module 8. The drawing shows, by way of example, an ECG module 9, a pulse oximeter 10 and a defibrillator 11. The defibrillator 11 is a therapy module and may additionally have sensors, such as a single-channel ECG. The connection between the sensors or therapy modules 9, 10, 11 (and other sensor or therapy modules—not shown) and the telemedicine module 8 can either be made by wire or wirelessly by means of a suitable radio or infrared link, examples which may be made in this case being Bluetooth or WLAN links.

The telemedicine module has a touchscreen, shown schematically at 12, and it is also possible for a video camera 13 and a headset 14 (headphones and microphone) to be connected to this. The connection to the video camera 13 and the headset 14 can again be made by wire or wirelessly. Power is supplied to the telemedicine module 8 preferably independently by means of a rechargeable battery, which preferably allows an adequate operating time of at least a few hours.

The telemedicine module 8 preferably has an internal database and a suitable computer which can provide the cabin crew with assistance in dealing with medical incidents and emergencies even if the telemedicine module 8 is operating independently and without a data link to a ground station 6. For example, stored instructions, video sequences or the like can be used to propose measures for attaching or handling sensors, measures for safeguarding vital functions or the like. In addition, the telemedicine module 8 can use the medical data captured by means of the sensors or input manually by the cabin crew using the touchscreen 12 to submit proposals for further action which are stored in the database.

Preferably, however, the telemedicine module 8 is not operated independently, but rather the WLAN 2 and the satellite link 3, 4, 5 are used to set up a data link to the ground station 6. The latter in turn preferably uses the Internet 7 to ensure a data link to a telemedicine service station—indicated schematically at 15—which is staffed by at least one doctor.

Preferably, the data link between the telemedicine module 8 and the telemedicine service station 15 is effected in encrypted form insofar as the data link is set up using data links or networks which are accessible to third parties. When the link has been set up, it is first of all possible for medical data captured by the telemedicine module 8, for example, to be sent to the doctor in the telemedicine station 15 and evaluated there. Using the camera 13, the doctor in the telemedicine station 15 can be shown the state of the patient or can visually check the correct performance of diagnostic or therapeutic measures by the cabin crew. The headset 14 can be used to interchange audio information.

If there is no provision or no further need for video transmission, the telemedicine module 8 can change to the second mode of operation. In this mode of operation, there is either now only a much lower bandwidth kept over the entire transmission line to the telemedicine station 15, or alternatively it is possible for discontinuous data transmission to take place in packet mode, particularly when only vital data for the patient which are captured by the telemedicine module 8 need to be continually transmitted to the telemedicine station 15. Since these data are buffer-stored in the telemedicine module 8, this generally non-time-critical data transmission is not adversely affected by a data transmission in packet mode.

The telemedicine module 8 preferably has a label printer which can print a label band—indicated at 16—which can be placed around the patient's wrist, for example. The printed label is used to identify the data record which is created about the medical incident and which is kept in a database in the telemedicine station 15 on the ground. If the patient is taken to a hospital, for example, when the aircraft has landed, the hospital can use an appropriate reader to read the code from the label 16 and in this way is provided with authorized access to the relevant data record in the database in the telemedicine station 15. The relevant data about the incident are then immediately available to the hospital, and at the same time unauthorized access by third parties is made more difficult.

The data link between the telemedicine module 8 and the ground station 6 and also the telemedicine station 15 can also be used, during or outside medical incidents, to perform a self-test on the telemedicine module 8, to perform a software update or the like.

1. An apparatus for capturing or evaluating medical data on board a means of transport, comprising:
at least one interface configured to capture or input medical data; and
a device configured to transmit medical data, audio data, or video data from the apparatus to or from a ground station situated outside the means of transport via a data transmitter,
wherein the apparatus has a first and second mode of operation for transmitting data to or from the ground station, the first mode of operation designed to use a first bandwidth sufficient for video transmission on the data transmitter and the second mode of operation designed to use a second bandwidth narrower than the first bandwidth or a discontinuous data transmission mode on the data transmitter.

2. The apparatus according to claim 1, wherein the at least one interface comprises connections for sensors that measure vital functions of the body.

3. The apparatus according to claim 1, wherein the at least one interface is further configured to capture audio or video data or receive input of data by an ancillary person.

4. The apparatus according to claim 1, wherein the device comprises a wireless or wired network connection for connecting to a network in the means of transport.

5. The apparatus according to claim 1, wherein the first bandwidth is at least 64 kBit/s.

6. The apparatus according to claim 1, wherein the second bandwidth is at least 16 kBit/s.

7. The apparatus according to claim 1, wherein the apparatus is configured to automatically use the first mode of operation when a video transmission is performed.

8. The apparatus according to claim 1, further comprising an identification device which, when the first mode of operation is used, identifies the apparatus to an operator of the data transmitter and requests an appropriate bandwidth.

9. The apparatus according to claim 1, wherein the apparatus has a third mode of operation in which no data transmission from or to a ground station takes place.

10. The apparatus according to claim 1, further comprising a label printer.

11. A system for capturing or evaluating medical data on board a means of transport, comprising:
an apparatus according to claim 1 on board the means of transport;
a network configured to transmit data from the apparatus to a first transmission/reception device in the means of transport on board the means of transport;
a ground station having a second transmission/reception device designed for data communication with the first transmission/reception device in the means of transport;
devices in the ground station for data communication with a device configured to provide telemedical services.

12. A system according to claim 11, wherein the network on board the means of transport is a wireless network.

13. A system according to claim 11, wherein data communication between the first transmission/reception device in the means of transport and the ground station is effected using a satellite link.

14. A system according to claim 12, wherein data communication between the first transmission/reception device in the means of transport and the ground station or data communication with the device configured to provide telemedical services is effected in encrypted form.

15. A system according to claim 11, further comprising a database configured to store transmitted medical data, the database being accessible to third parties which have an authorization via a data network.

16. The apparatus according to claim 1, wherein the first bandwidth is 64 to 128 kBit/s.

17. The apparatus according to claim 1, wherein the first bandwidth is at least 96 kBit/s.

18. The apparatus according to claim 1, wherein the second bandwidth is at least 32 kBit/s.

19. The apparatus according to claim 1, wherein the second bandwidth is 32 to 64 kBit/s.

20. The system according to claim 12, wherein the wireless network is a W-LAN.

21. The system according to claim 11, wherein data communication with the device configured to provide telemedical services is effected via the Internet.
